(19) United States
(12) Patent Application Publication
(10) Pub. No.: US 2001/0039442 A1
(43) Pub. Date: Nov. 8, 2001

(54) Headache relief device

(76) Inventors: Sal Gorge, Boca Raton, FL (US); Nancy (O?apos;Kuhn) Gorge, Boca Raton, FL (US)

Correspondence Address: Robert M. Downey, ROBERT M. DOWNEY, P.A., 150 E. Palmetto Park Road, Suite 350, Boca Raton, FL, 33432, US

(21) Appl. No.: 09/849,798

(22) Filed: May 4, 2001

Related U.S. Application Data
(60) Provisional application No. 60/202,524, filed on May 6, 2000.

(51) Int. Cl. A61F 7/00

(52) U.S. Cl. 607/109; 607/112; 607/114

(57) ABSTRACT

A device for treating headaches includes an eye mask which may be formed and configured to cover both eyes or, alternatively, one eye of the user. The eye mask is provided with straps which are secured behind the head to hold the eye mask in the desired, operative position. The inner facing side of the eye mask is provided with soft, compliant membrane portions forming rear walls of left and right containers. The containers are provided with a fill cap which removes to facilitate filling of a liquid or gel through an opening and into an interior chamber in direct, temperature transferring relation to the respective compliant membrane portions. The liquid or gel may be brought to a desired temperature just prior to filling or, alternatively, when filled within the reservoir. The compliant membrane portions are provided with a concave configuration for congruent receipt with the periphery of the eyes and the upper and lower eye lids when the user's eyes are closed. When secured to the user's head, the mask blocks light while the compliant material rear walls of the containers apply a warm, ambient or cool compress to the closed eyes.

### BACKGROUND OF THE INVENTION

This application is based on provisional patent application Ser. No. 60/202,524 filed on May 6, 2000.

### FIELD OF THE INVENTION

The present invention relates to a headache relief device and, more particularly, to an eye mask including one or more chambers adapted to be filled with a temperature variable fluid substance and one or more membranes adapting for mating engagement with the closed eyelids and surrounding eye cavities for applying a warm, ambient or cool temperature compress to a person's eyes in order to alleviate headache pain.

### DISCUSSION OF THE RELATED ART

At one time or another, virtually everyone has experienced the pain and discomfort of a headache. However, headaches occur more frequently in some individuals, particularly if they have a family history of this problem. Also, the severity of headache pain will vary depending on the individual and the type of headache. There are many causes for headaches, as well as numerous factors which may trigger the onset of a headache. The specific underlying cause of the headache will naturally determine the type of headache. Generally there are five primary headache types, namely: migraine headaches; hormonal headaches; stress-related headaches; sinus headaches; and cluster headaches. Migraines and cluster headaches tend to be a chronic condition and are often more severe than the other types of headaches.

Depending upon the severity and type of headache, various means of treating the pain may be used. The most common method for treating a headache involves the use of over-the-counter pain relievers such as aspirin, acetaminophen, ibuprofen, naproxen, and ketoprofen. In cases of more sever and chronic headaches, a doctor may prescribe any of various drugs, including pain killers and abortive drugs which target serotonin receptors to stop or reduce the severity of an attack. Most new prescription drugs for severe headache pain fall into the abortive class and are commonly referred to as “triptans.” In case of severe migraine headache pain, use of a prescribed drug may be the only means of immediate relief. However, many headaches can be treated without the use of drugs and, in less severe cases, it may be best to first attempt use of alternative treatments to avoid becoming dependent on more powerful, potentially addictive drugs. Moreover, if prescription drugs are used too often, they may become less effective in treating the symptoms of headache.

Alternative treatments for headaches include such procedures as acupuncture, BOTOX injections, and chiropractic adjustments. While these treatments have been proven to be effective in some cases, they are expenses and they are not readily available when the onset of a headache occurs. Other alternative treatments involve the use of natural herbs and are not scientifically proven to alleviate pain and other symptoms associated with the various types of headaches. Moreover, there is growing concern in the healthcare industry over the misuse and/or excessive use of herbal medicines which may lead to serious side effects, particularly if the patient is allergic.

Accordingly, there is an urgent need in the healthcare field for a safe, low cost means for the immediate treatment of pain and other symptoms associated with headache. More particularly, there is an urgent need for a device which is easy to use and which is adapted to apply warm, ambient or cool temperature to a patient's closed eyelids and surrounding eye cavity in order to alleviate headache pain.

### SUMMARY OF THE INVENTION

The subject invention is directed to a device for treating human ailments, with particular attention to headaches and is based on the empirical knowledge of the inventor. The device consists of a soft material mass (e.g., felt, velvet, silk or the like) to be disposed around the head with the capabilities of covering the eyes to block light and allowing the application of warm, ambient or cool compress around the periphery of the eyes and to the closed eye lids. The temperature applied to each closed eye may be individually controlled. Additionally, the pressure applied to the closed eye(s) may be controlled for maximum effect and comfort via the adjustment of straps which secure behind the wearer's head with the use of hook and loop fasteners.

### BRIEF DESCRIPTION OF THE DRAWINGS

For a fuller understanding of the nature of the present invention, reference should be made to the following detailed description taken in conjunction with the accompanying drawings in which:

FIG. 1 is a top front perspective view illustrating a headache treatment device to be worn on a user's head in covering relation to the eyes, in accordance with a preferred embodiment of the present invention;

FIG. 2 is a rear top perspective view of the headache treatment device of FIG. 1;

FIG. 3 is an isolated top plan view taken along the plane indicated by the arrows 3-3 in FIG. 2;

FIG. 4 is an isolated view, in partial cross-section, taken along the plane of the line 4-4 of FIG. 3 showing a liquid-filled chamber of one of two containers on a eye mask portion of the device;

FIG. 5 is a front perspective view of the mask of the present invention, in accordance with an alternative embodiment thereof;

FIG. 6 is a particularly exploded rear perspective view of the mark of FIG. 5, illustrating a pair of open pockets for receipt of containers therein;

FIG. 7 is a top perspective view of the container shown in FIG. 6;

FIG. 8 is a side elevation, in partial section, illustrating the concave configuration of the rear side of the container;

FIG. 9 is a cross sectional view showing the container fitted within the pocket of the face mask;

FIG. 10 is a top front perspective view of a cooling device which is adapted to be placed about the forehead or neck of the user to apply a warm, ambient or cool compress to the forehead or neck, in accordance with another embodiment of the invention;

FIG. 11 is an isolated sectional view taken along the plane of the line 11-11 of FIG. 10, illustrating a plug on the rear end of the device of FIG. 10 for filling a liquid, such as water, within an interior chamber thereof.

Like reference numerals refer to like parts throughout the several views of the drawings.

### DETAILED DESCRIPTION OF THE PREFERRED EMBODIMENTS

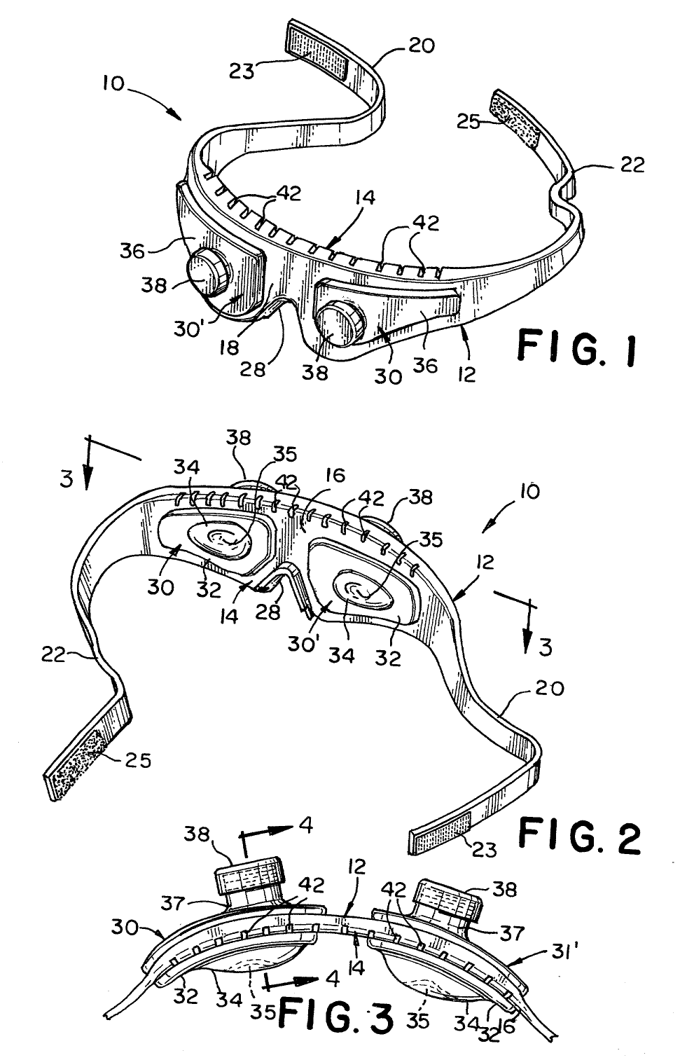

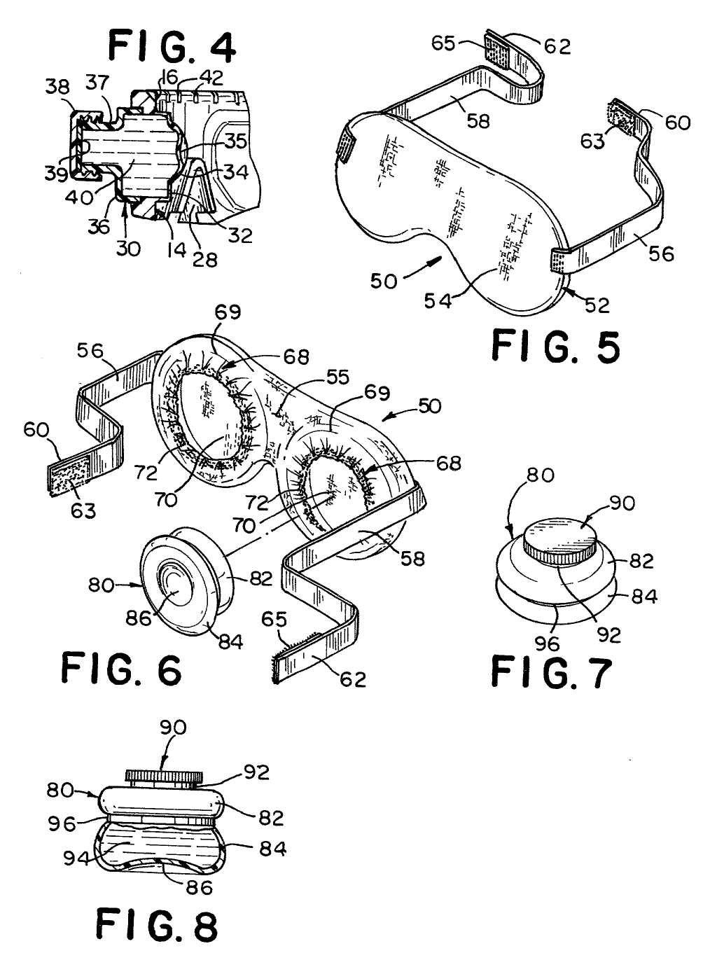

Referring initially to FIGS. 1 through 4, a headache treatment device is shown in accordance with a preferred embodiment of the invention and is generally indicated as 10. The device 10 is defined primarily by an eye mask 12 formed of a soft, flexible material such as an elastomeric plastic or latex which is adapted to wrap about and conform to the exterior configuration of a user's head. The eye mask 12 includes an inner side 16 which is smooth for comfortable engagement against the skin of the face and sides of the wearer's head. The eye mask 12 further includes an outer side 18 which remains exposed when the device 10 is worn on a person's head, in covering relation to the eyes.

To secure the device 10 to a wearer's head, head straps are provided, including a right head strap 20 and a left head strap 22 which are integral with and extend from the eye mask 12. In a preferred embodiment, the left and right head straps 20, 22 are formed of the same flexible material as the eye mask 12. To facilitate adjustable fastening of the straps 20, 22 about the head, releasable attachment means are provided on the strap ends. In a preferred embodiment, hook and loop fastening means are provided, including a hook material 23 on the distal end portion of the strap 20 and a loop material 25 on the distal end portion of the strap 22. A nose bridge 28 rests on the wearer's nose to comfortably support and maintain the eye mask 12 in covering relation to the eyes, with the distal ends of the straps 20, 22 secured to one another at the back of the head.

The device 10 further includes a left container 30 and a right container 30′ fitted to the eye mask 12. Each container 30, 30′ includes a rear side 32 which is integral with the eye mask 12. The rear side 32 of each container protrudes out from the inner side 16 of the eye mask 12 and is preferably made of the same soft flexible material. A bulbous portion 34 is provided on the rear side 32 of each container 30, 30′. The bulbous portion 34 is sized and configured to mate against a respective left or right eye cavity on the facial exterior of the wearer. A concave relief 35 on each bulbous portion 34 mates with the closed eyelid and conforms with the bulging shape of the anterior portion of the eyeball, thereby relieving excess pressure on the cornea and optic nerves below the eyelid. The containers 30, 30′ are further provided with a rigid front side 36 which is attached and sealed to the outer side 18 of the eye mask 12 in covering and closing relation to a respective left and right cavities formed in the eye mask 12. A neck 37 is integral with the front side 36 on each of the containers 30, 30′ and extends outwardly from the front side 36 terminating at an open end to facilitate filling and emptying of liquid which is normally contained within an interior chamber 40 formed in the cavity enclosed by the rear side 32 of the eye mask 12 and the front side 36 of the containers 30, 30′. A cap 38 is removably attachable to the open end of the neck of each of the respective containers 30, 30′ to thereby cover the opening. A rubber seal 39 on the inner side of the cap provides a water tight seal so that the fluid contained in the interior chamber 40 does not leak when the caps 38 are secured to the open ends of the neck. The top edge of the eye mask 12 is provided with a plurality of spaced notches 42 which promote flexibility and bending of the eye mask 12 to conform to the curvature of the wearer's face.

In use, the caps 38 are removed from the respective containers 30, 30′ to facilitate filling the interior chambers 40 with a fluid. In a preferred embodiment, the chambers 40 are filled with tap water or saline solution. It is preferable that the fluid be cooled or warmed just prior to filling into the interior chambers 40. Specifically, if it is desired to apply a cold compress to the closed eyes, the fluid should be cooled to a temperature preferably between 40° F. and 55° F. Alternatively, if it is desired to apply warmth to the closed eyes, the liquid should be warmed to a temperature which is comfortable to the wearer, and yet which will not cause burning and/or harm to the skin or eyes. It is important to note that the membrane of material forming the rear side 32 and bulbous portion 34, including the concave relief 35, of each container 30, 30′ is of a thickness and material composition which is suitable to permit heat transfer between the wearer's skin and the fluid filled within the respective chambers 40.

Referring to FIGS. 5 through 9, an alternative embodiment of the headache treatment device of the present invention is shown and is generally indicated as 50. The device 50 includes a soft, pliable face mask 52, similar to a sleep mask, which may be formed of any suitable materials which are comfortable to the skin and which are cosmetically appealing including, but not limited to, felt, velvet, silk or cotton. The mask 52 includes a front side 54 and a rear side 56. Straps 56 and 58 extend from opposite sides of the mask to free distal ends 60, 62 which are provided with hook and loop fastening means 63, 65 for adjustable, releasable attachment of the distal ends 60, 62 on the rear of the wearer's head, thereby facilitating adjustable attachment of the mask 52 in covering relation to the wearer's eyes. It should be noted that the mask 52 may be formed and configured as an eye patch, for covering only one eye, if it is desired to maintain visibility with the other eye.

The rear side 55 of the mask 52 is provided with open pockets 68 which are stitched or secured about their periphery 69 to the rear face 56 of the mask. The pockets 68 are preferably formed of a soft fabric, similar to the mask 52, and include an opening 70 surrounded by an elastomeric band 72. It should be noted that the mask device is configured as an eye patch, for covering one eye, only one pocket 68 is provided on the rear side 52.

FIGS. 3-6 illustrate a container 80 used in conjunction with the mask 52 for applying a warm, ambient or cool compress to the closed eyes of the user. Each container 80 is sized and configured for receipt within a respective one of the pockets 68 and includes a front portion 82 and a rear portion 84. In a preferred embodiment, the front portion is formed of a rigid material, such as a rigid plastic, while the rear portion 84 is formed of a compliant or resilient material such as a soft rubber. The softer, more compliant material of the rear portion 84 is particularly suited to make contact with the user's facial area and, more particularly to the periphery of the eyes and the outer surface of the eye lids, when the user's eyes are closed. Moreover, the compliant material of the rear portion 84 is provided with a concave area 86 on the rear end of the container which is adapted to assume the outer surface configuration of the closed eye, in a manner which permits a congruent, mating engagement of the concave region 86 with the closed eye lid and eye periphery.

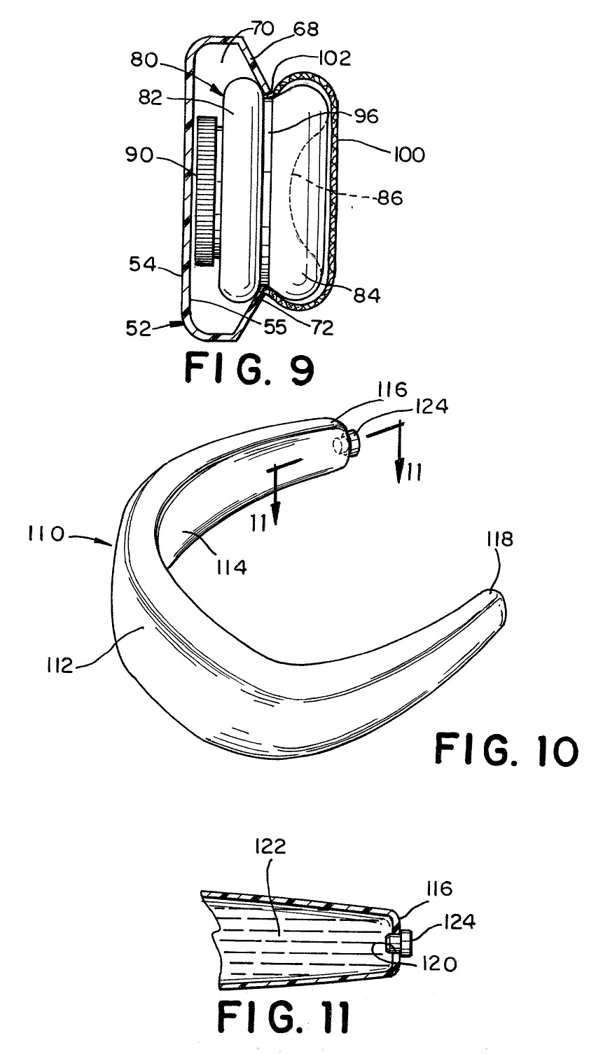

Referring to FIGS. 7 and 8, the container 80 is shown to include a fill cap 90 which may be threadably secured to an annular collar 92 surrounding an opening of the container which communicates with an interior reservoir 94. The fill cap 90 forms a seal when closed and allows filling and emptying of the reservoir 94 when open. The front portion 82 of the container is formed of a more rigid material and is bonded to the compliant material of the rear portion 84 during the manufacturing process. An annular groove 96 is provided at the juncture between the front portion 82 and rear portion 84 of the container and is particularly adapted for receipt of the elastomeric band 72 surrounding the opening 70 of a respective one of the pockets 68 so that when the container is fitted within the pocket, as shown in FIG. 9, the annular band 72 closes in grasping relation within the annular groove 96. More specifically, to insert and remove the container 80 from within a respective one of the pockets 68, the annular band 72 is stretched to enlarge the size of the opening 70. This permits placement and removal of the front portion 82 of the container within the pocket, as seen in FIG. 9, so that the fill cap 90 and front portion 82 are captivated within the pocket 68. The elastomeric band 72 can then be released and allowed to close upon the annular groove 96 to hold the container within the pocket so that the rear compliant portion 84 is disposed outwardly of the pocket for mating engagement with the user's eye.

A cover may be further provided formed of a soft material may be provided for covering the rear portion 84, as shown in FIG. 9, in instances where the temperature of the fluid contained within the reservoir is excessively cold or hot, to the extent that it causes discomfort to the user. In this manner, the cover 100 may be used to provide a barrier of protection against excessive temperatures. The cover 100 is fitted to the rear portion 84, in the manner similar to the securing of the pocket 68 over the front portion 82. More specifically, the cover 100 includes an elastomeric band 102 which is secured within the annular groove 96 to hold the cover 100 in covering relation to the rear portion 84.

FIGS. 10 and 11 illustrate a compress head wrap 110 which is particularly adapted to be fitted around the wearer's forehead or the back of the neck. Similar to the container 80, as described above, the head wrap 110 includes a front portion 112 formed of a rigid material, such as plastic, and a rear portion 114 formed of a compliant material, such as a soft rubber similar to that used for the container 80. The compliant material 114 is particularly suited for contact with the skin of the wearer's head, and particularly the forehead and the back of the neck. The head wrap 110 includes opposite ends 116, 118 which are adapted to extend around the sides of the head or neck in a manner which holds the head wrap device 110 in place. More specifically, the head wrap device 110 is formed and configured to partially encircle the head or neck and to apply slight pressure around the sides of the head or neck to hold the head wrap device 110 in place. One of the ends 116 is provided with a fill opening 120 communicating with an interior reservoir 122 for filling a liquid or gel therein. Similar to the container, the temperature of the liquid or gel filled within the reservoir 122 can be controlled, either by heating or cooling the liquid/gel prior to filling, or alternatively, heating or cooling the liquid/gel when contained within the reservoir. In both the embodiments, having either the container 80 or the head wrap 110, the respective reservoirs of these devices may be filled with liquid or gel and then placed in a refrigerator or freezer to cool the contents in the reservoir in a manner which transfers the temperature to a compliant material 114 or 84 of the respective devices. The opening 120 of the head wrap device 110 is closed with a cap 124 to contain the contents of the reservoir 122. The cap 124 is specifically positioned in a manner which will not cause discomfort to the wearer when the device 110 is fitted to the wearer's head or neck, while lying on a couch or sitting in a chair. More specifically, the cap 120 is located in an area which avoids contact with the user's head or a head rest surface, such as a pillow, cushion or back rest of a chair or couch.

While the instant invention has been shown and described in accordance with preferred and practical embodiments thereof, it is recognized that departures from the instant disclosure are contemplated within the spirit and scope of the invention as set forth in the following claims and as interpreted under the doctrine of equivalents.

Now that the invention has been described,

What is claimed is:

1. A device for treating a patient suffering from a headache, said device comprising:
an eye mask structured and disposed for covering at least one eye of the patient to define an operative position, said eye mask including an inner side and an outer side;
means for removably securing said device to the patient's head to hold said eye mask in said operative position;
a membrane positioned on said inner side of said eye mask and formed of a soft, compliant, heat transferable material; and
a container carried on said eye mask and including:
an interior chamber structured and disposed for containing a variable temperature fluid substance therein in heat transferring relation with said membrane; and
an opening with a removable cap for covering said opening, said opening being disposed in fluid communication with said interior chamber to facilitate filling and emptying the fluid substance in the interior chamber.

2. The device as recited in claim 1 wherein said means for removably securing said device to the patient's head includes strap means fitted to said eye mask and being structured to extend about the patient's head.

3. The device as recited in claim 2 wherein said strap means includes opposite first and second straps extending from opposite sides of said eye mask and including free distal end zones and means for removably securing said distal end zones to one another for holding the straps securely about the wearer's head while holding the eye mask in the operative position.

4. The device as recited in claim 3 wherein said means for removably securing said distal end zones of said straps includes hook and loop fastening means.

5. The device as recited in claim 2 further comprising means for removably attaching said container to said eye mask.

6. The device as recited in claim 2 wherein said membrane includes a protruding bulbous portion.

7. The device as recited in claim 6 wherein said protruding bulbous portion of said membrane includes a concave relief, said bulbous portion and said concave relief being structured and disposed for congruent mating engagement with the closed eyelids and surrounding eye cavity of the patient when said eye mask is held in said operative position.

8. The device as recited in claim 1 wherein said eye mask is structured and disposed for covering both eyes of the patient.

9. The device as recited in claim 8 wherein said device includes two of said membranes, including a left membrane and a right membrane, said left and right membranes being structured and disposed for corresponding mating engagement with the closed eyelids and surrounding eye cavities of the left and right eyes of the patient.

10. The device as recited in claim 9 wherein said device comprises two of said containers, including a left container and a right container, said left and right containers each including said interior chamber communicating in heat transferring relation with a corresponding one of said left and right membranes.

11. The device as recited in claim 10 wherein said left and right membranes each comprise said bulbous portion and said concave relief for congruent mating engagement with the closed eyelids and surrounding eye cavities of the left and right eyes of the patient.

12. A device for treating a patient suffering from a headache, said device comprising:
an eye mask structured and disposed for covering the left and right eyes of the patient to define an operative position, said eye mask including an inner side and an outer side;
means for removably securing said device to the patient's head to hold said eye mask in said operative position;
a left membrane and a right membrane each positioned on said inner side of said eye mask and formed of a soft, compliant, heat transferable material;
said left and right membranes being structured and disposed for congruent mating engagement with the closed eyelids and surrounding eye cavities of the left and right eyes of the patient; and
a container carried on said eye mask and including:
an interior chamber structured and disposed for containing a variable temperature fluid substance therein in heat transferring relation with said left and right membranes; and
an opening with a removable cap for covering said opening, said opening being disposed in fluid communication with said interior chamber to facilitate filling and emptying the fluid substance in the interior chamber.

13. The device as recited in claim 12 wherein said means for removably securing said device to the patient's head includes strap means fitted to said eye mask and being structured to extend about the patient's head.

14. The device as recited in claim 13 wherein said strap means includes opposite first and second straps extending from opposite sides of said eye mask and including free distal end zones and means for removably securing said distal end zones to one another for holding the straps securely about the wearer's head while holding the eye mask in the operative position.

15. The device as recited in claim 14 wherein said means for removably securing said distal end zones of said straps includes hook and loop fastening means.

16. The device as recited in claim 12 wherein said left and right membranes each include a protruding bulbous portion.

17. The device as recited in claim 16 wherein said protruding bulbous portion of said left and right membranes includes a concave relief, said bulbous portion and said concave relief being structured and disposed for congruent mating engagement with the closed eyelids and surrounding eye cavities of the left and right eyes of the patient when said eye mask is held in said operative position.

18. The device as recited in claim 12 wherein said device includes a plurality of said containers, including a left container and a right container, said left and right containers being independent of one another and each including said interior chamber, said interior chamber of each of said left and right containers communicating with a corresponding one of said left and right membranes.
